(19) United States
(12) Patent Application Publication
(10) Pub. No.: US 2008/0275109 A1
(43) Pub. Date: Nov. 6, 2008

(54) NEUTRALIZING AGENT FOR VACUOLATING TOXIN

(75) Inventors: Kinnosuke YAHIRO, Chiba-shi (JP); Motoyuki Tagashira, Moriya-shi (JP); Toshiya Hirayama, Nagasaki-shi (JP); Masatoshi Noda, Yotsukaido-shi (JP)

Correspondence Address: BROWDY AND NEIMARK, P.L.L.C.;624 NINTH STREET, NW, SUITE 300, WASHINGTON, DC, 20001-5303, US

(73) Assignees: Asahi Breweries, Ltd., Chuo-ku (JP); Toshiya HIRAYAMA, Nagasaki-shi (JP); Masatoshi NODA, Yotsukaido-shi (JP)

(21) Appl. No.: 12/041,550

(22) Filed: Mar. 3, 2008

Related U.S. Application Data
(63) Continuation of application No. 10/574,482, filed on Apr. 3, 2006, now abandoned.

(30) Foreign Application Priority Data
Oct. 2, 2003 (JP) 2003-344433

(51) Int. Cl. A61K 31/353; C07D 311/04

(52) U.S. Cl. 514/456; 549/399

(57) ABSTRACT

A drug, quasi drug, food or drink product containing a proanthocyanidine derived from hop, hop bract or apple has the effect of neutralizing or attenuating a vacuolacting toxin produced by Helicobacter pylori, thus preventing, preventing recurrence or treating digestive diseases in which Helicobacter pylori participates, as well as killing Helicobacter pylori, while providing few side effects and being free of risk of the occurrence of a tolerant strain.

### TECHNICAL FIELD

The present invention relates to a neutralizing agent, a drug, a quasi drug, a food or drink product containing, as an active ingredient, proanthocyanidines having an effect of preventing, preventing recurrence or treating digestive diseases participated by Helicobacter pylori, and having an effect of neutralizing (attenuating) a vacuolating toxin produced by Helicobacter pylori, particularly preferably proanthocyanigines originating in apple or hop bract.

### BACKGROUND ART

Hop (Humulus lupuls) is a perennial plant of a Cannabaceae family and the hop cone (a ripe unfertilized female flower) is generally called hop. The hop comprises, in addition to the cones, leaves, bines, roots and the like. A lupulin part (a yellow granule formed in the root of internal bracts of the hop cone) existing in the hop cone is a source of bitterness and perfume and it is an important beer material along with yeast and malt in beer brewing. The hop can be used as a sedative drug and an anti-aphrodisiac in folk remedies. The hop bracts are formed by removing the lupulin part from the hop cone, and these bracts are useless. If circumstances require, these bracts are removed in beer brewing to be byproducts. In this case, the hop bracts are used as fertilizer. However, since special effective use is not found, it is hoped to develop a method having a high additional value for using the bracts.

In Japanese patent literatures 1, 2, 3, 4, 5, and 6, it is recognized that hop, particularly the polyphenols derived from hop bracts have antioxidant action, sparkle-stabilizing action of sparkling malt drinks, anticaries action, deodorant action, antimetastatic action of cancer cells, and topoisomeraze-inhibiting action. Further, in Japanese Patent literature 7, it is recognized that hop has protein toxin-neutralizing effect of RNA N-glycosidase activity and ADP ribosyltransferase activity.

However, as to proanthocyanidines obtained from hop, no cases proving neutralization (attenuating) effect of proteotoxin have been found hitherto.

Helicobacter pylori (abbreviated as pylori hereinafter) is a gram-negative bacillus having a spiral form. Since Warren and Marshal had reported the presence (non-patent literature 1), it becomes apparent that many diseases of digestive organ such as acute gastritis, chronic gastritis, gastric ulcer and duodenal ulcer are influenced by the bacillus (non-patent literatures 2, 3 and 4). In addition, since 90% or more of patients suffering from stomach cancer are bacterial carriers of pylori, there is high possibility that pylori participates in development of the stomach cancer. WHO reported in 1994 “it is obvious that pylori is a carcinogenic factor of the stomach cancer”.

As a disease factor producing pylori, urease, catalase, lipopolysaccharide (LPS) and the like have been reported. Lately, it has been appeared that animal models suffered from gastritis initiates denaturation of vacuolation in mucosae of the stomach by giving a vacuolating toxin (Vac A) alone (non-patent literature 5). It is rapidly recognized that the vacuolating toxin is a main (or dominant) virulence factor of pylori.

Hitherto, in the treatment of ulcerative diseases such as gastric ulcer and duodenal ulcer, anti-ulcer agents such as sofalcone and plaunotol; proton pump inhibitors (PPI) such as omeprazole and lansoprazole acidity secretion depressants (H2 blocker) such as famotidine and cimetidine have been used. However, these agents do not have effects for inhibiting the pylori proliferation, but are symptomatic therapy agents for ulcerative diseases. Accordingly, even though these agents cure the ulcerative diseases, pylori remain in the stomach. There is defect that the return percent is high 80-90% within one year.

To resolve the defect, in addition to the symptomatic therapy, a treatment method for removing the pylori has been reported, and antibiotic drugs such as amoxicillin, clarithromycin, metronidazole and tinidazole have been clinically used. Nowadays, a treatment method for removing bacteria is mainly using together three agents of a proton pump inhibitor and two antibiotic drugs.

However, the treatment method using together three agents has some clinical problems such as long-term administration of relatively large amount of agents, side effects of agents and replacement of bacteria. The use of antibiotic drugs brings bacteria destruction, and a large amount of vacuolating toxins which is a disease factor producing pylori may be produced. Further, when a large amount of antibiotic drugs is used, more strength resistant bacteria may be produced. Considering the above knowledge, in spite of the method broadly used, it is difficult to say that the new method for using together three agents is an ideal method.

In Japan, the generation of 40 or more ages has a high percentage of pylori infection, and shows higher attack percentages of ulcerative diseases and stomach cancer than those of Europe and America. If a treatment method not having side effects and problems of resistant bacteria is found, the industrial value is very high.
Patent literature 1: Japanese Patent Laid-open publication No. 09-2971
Patent literature 2: Japanese Patent Laid-open publication No. 09-163969
Patent literature 3: Japanese Patent Laid-open publication No. 09-295944
Patent literature 4: Japanese Patent Laid-open publication No. 10-25232
Patent literature 5: Japanese Patent Laid-open publication No. 2000-327582
Patent literature 6: Japanese Patent Laid-open publication No. 2001-39886
Patent literature 7: WO02/07826 Pamphlet
Non-patent literature 1: Lancet, 1273-1275 (1983)
Non-patent literature 2: Med. J. Aust., 142, 436 (1985)
Non-patent literature 3: Gastroentrology 102, 1575 (1992)
Non-patent literature 4: N. Engl. Med., 328, 308 (1993)
Non-patent literature 5: Infect. Immun., 63, 4154-4160 (1995)

### PROBLEMS TO BE SOLVED BY THE INVENTION

Accordingly, the present invention aims to provide a drug, a quasi drug, a food or drink product having an effect of preventing, preventing recurrence or treating digestive diseases, in which Helicobacter pylori participates. The inventors of the present invention, considering these facts, have tried to solve the problems by finding the factor for attenuating a vacuolating toxin produced by Helicobacter pylori without killing the bacteria. If the factors attenuating the vacuolating toxin are found, the use is industrially valuable.

### DISCLOSURE OF INVENTION

The inventors of the present invention have studied earnestly and found that a kind of polyphenols contained in hop and apples effectively attenuate the vacuolating toxin produced from pylori, and the invention has been made. The large amount of polyphenol is particularly contained in immature apple fruit and hop bracts.

The polyphenol contained in hop is adsorbed on resin showing affinity for polyphenol such as styrene-divinylbenzene. When the polyphenol is treated with an ultrafiltration membrane having 1000 or more of fraction molecular weight, it shows the property not penetrating the membrane. When the polyphenol is heated in an alcohol solution of about 5% hydrochloric acid, it is hydrolyzed to produce cyanidine. The polyphenol is considered as a proanthocyanidine. The proanthcyanidine shows a chromatogram of FIG. 1 in GPC (gel permeation chromatography) analysis. On the other hand, it shows absorbance distribution of FIG. 2 in absorbance analysis. Moreover, the polyphenol contained in apples is adsorbed on a resin having affinity for polyphenol such as styrene-divinylbenzene. When the polyphenol is heated in an alcohol solution of about 5% hydrochloric acid, it is hydrolyzed to produce cyanidine. The polyphenol is considered as a proanthocyanidine.

Namely, the present invention relates to a neutralizing agent for a vacuolating toxin containing as an effective ingredient the proanthocyanidine, particularly derived from hop or apple.

As materials neutralizing the vacuolating toxin, 5-nitro-2-(3-phenylpropylamino) benzene acid and Phloretin and a part of polyphenols, which inhibit the current change on cell membrane produced by the vacuolating toxin, are reported by Tombola et al (Tombola F. et al., FEBS Lett. 543, 184-189 (2003)). However, it is reported that, in these systems, the materials inhibiting the current change on cell membrane are not concerned with inhibition of cell vacuolating with vacuolating toxin or neutralization of a cell toxin. Moreover, though the compounds shown in these literatures are polyphenols, all of the compounds are not proanthocyanidines.

Accordingly, a technique for obtaining a nontoxic vacuolating toxin with the proanthocyanidines originated from plants, particularly preferable hop or apple has not been reported.

### BRIEF DESCRIPTION OF DRAWINGS

FIG. 1 shows an analytical result of GPC (gel penetration chromatography) of proanthocyanidines originated from hop.

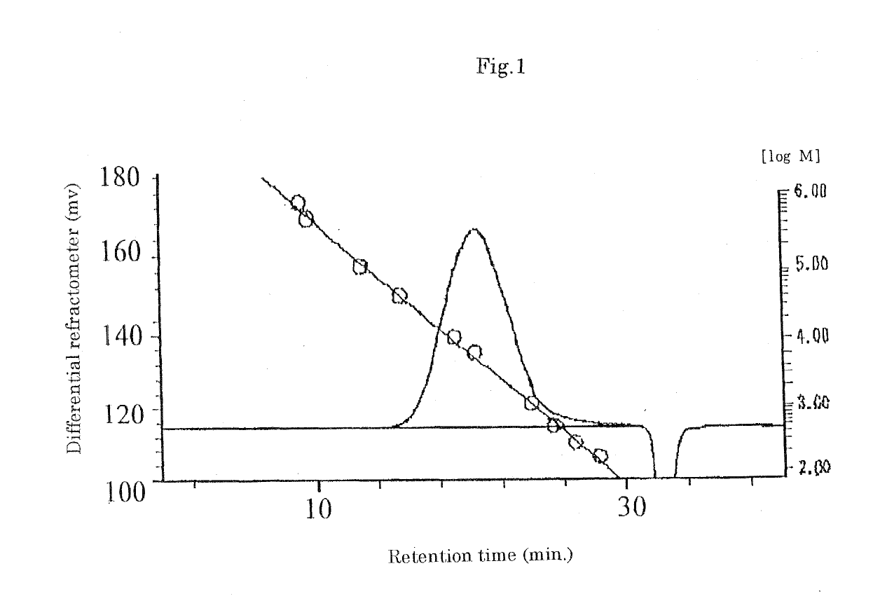

FIG. 2 shows absorbance distribution of proanthocyanidines originated from hop.

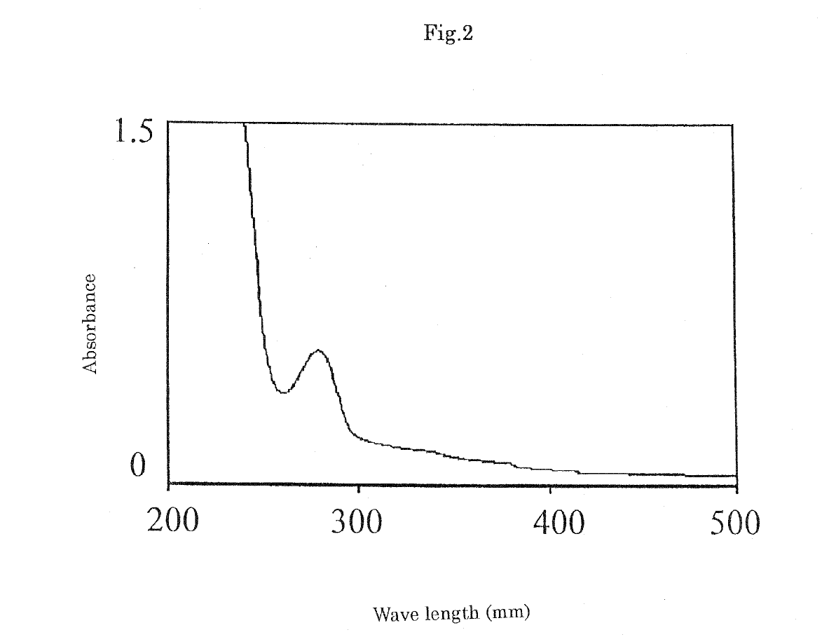

FIG. 3 shows an analytical result of HPLC of proanthocyanidines originated from hop.

FIG. 4 shows that a vacuolating toxin changes into a nontoxic material in a cultured cell of a human stomach cancer cell AZ-521 (Example 13).

FIG. 5 shows that a vacuolating toxin changes into a nontoxic material in a cultured cell of a human renal cancer cell G401 (Example 13).

FIG. 6 shows inhibition of incorporation of a vacuolating toxin into a cultured cell of a human stomach cancer AZ-521 (Example 14).

FIG. 7 shows inhibition of incorporation of a vacuolating toxin into a cultured cell of a human renal cancer G401 (Example 14).

### BEST MODE FOR CARRYING OUT THE INVENTION

As to the materials of a neutralizing agent for vacuolating toxin, in addition immature apple fruit, hop bines and bracts are preferable. Particularly, the whole apple or hop can be used without separating into parts.

Hop bracts are obtained by removing the lupulin part from the hop cone. Usually after the hop cone is crushed, the lupulin part is sieved off to obtain the hop bracts. However, in recent beer brewing, in order to save trouble of removing the hop bracts by sieving, the hop cone is formed in a pellet form without removing the unusable bracts to obtain a hop pellet and use in beer brewing. Accordingly, as materials of the present invention, non-limited materials containing hop bines and bracts are used, and there is any problem to use the hop cone containing hop bracts or hop pellet.

The production for obtaining the neutralizing agent for a vacuolating toxin is that hop bines and bracts, the hop cone containing hop bracts or hop pellet, or a part of hop plants are used as raw materials, these materials are extracted with water, 80 v/v % or less of an aqueous alcohol solution, an aqueous solution of an organic solvent such as acetone or acetonitrile miscible with water. Preferable embodiment is an aqueous solution of 50 v/v % or less ethanol. The rate of materials and extraction solvent is preferably 1:20-100 (rate by weight). The extraction is preferably done at a temperature of 4-95° C. with stirring for 20-60 minutes. The extracted liquid is obtained by filtration and if necessary, auxiliary filter such as perlite may be used.

The obtained extraction liquid was usually concentrated, freeze-dried or splay-dried to remove solvent and obtain powder of the neutralizing agent for vacuolating toxin. Although such obtained neutralizing agent for vacuolating toxin may be used as it is, if necessary the purification may be increased by the following adsorption resin method.

The step may be omitted if such purification is unnecessary.

The above extraction liquid is treated with granular synthetic resin having affinity for polyphenols to concentrate the neutralizing agent for vacuolating toxin. This step may be done by passing the hop extraction liquid through the column filled with granular synthetic resin, thoroughly washing the column and eluting the neutralizing agent for vacuolating toxin. Further, the step may be done by dipping the granular resin in hop extraction liquid, and treating it by a batch process.

When the neutralizing agent for vacuolating toxin is adsorbed on the synthetic resin, after cooling the hop-extracted liquid to a room temperature of 15-30° C., if necessary, the concentration of organic solvent of the extracted liquid is preferably lowered previously by vacuum concentration for better adsorption efficiency. As the materials of the synthetic adsorption, hydroxy propyl dextran, hydrophilic vinyl polymer, styrene-divinyl benzene polymer, and the like may be used.

Then, the synthetic resin may be washed to increase the purification of the neutralizing agent for vacuolating toxin. As the solvent for washing, water or a 1-10 w/w % ethanol aqueous solution is preferably used. Preferably, the solvent quantity of 1-10 times of the resin may be used.

Followed by washing, the neutralizing agent for vacuolating toxin was eluted from the synthetic resin adsorbed polyphenol and the like. The solvent for elution may be such as hydrous alcohol, hydrous acetone, or hydrous acetonitrile. Particularly preferable example of the solvent is a 30 or more w/w % ethanol aqueous solution or ethanol. The solvent passing through the resin is preferably 2-6 times by weight of the resin.

The solvent may be concentrated from the eluting liquid by freeze-drying or splay drying to remove the solvent, and the neutralizing agent for vacuolating toxin may be obtained as powder. When the solvent is concentrated under reduced pressure, alcohol, acetone, acetonitrile or the like may be recovered to reuse. The used synthetic resin is washed with an 80 or more v/v % alcohol aqueous solution, about 0.05 N sodium hydroxide aqueous solution or the like, and may be used repeatedly.

Although thus obtained neutralizing agent for vacuolating toxin may be used, as it is, the following method using an ultrafilter serves the purification. However, since this step aims to raise the purification of the neutralizing agent for vacuolating toxin, if it is unnecessary, it may be omitted.

The neutralizing agent for vacuolating toxin obtained by the above-described method is dissolved in water or a mixture of organic solvent and water, and treated with a ultrafilter of 1000 or more of fraction molecular weight. The materials of the ultrafilter may be cellulose, cellulose acetate, polysulfone, polypropylene, polyester, polyethersulfone, PVDF and the like, and these materials may be used without particular limitation. The fraction molecular weight of 1000 or more may be used without particular problems. However, when the fraction molecular weight is too great, the yield becomes significantly small. When the fraction molecular weight is small, the treatment time becomes long. The optimum fraction molecular weight of the ultrafilter is about 5,000-50,000. Though the treatment depends on the kind of extract solvent, the rate of extract solvent and hop or hop bract, it is preferably to reduce the amount of the upper residue liquid to about 1/10- 1/100 of the beginning. Though the pressure is dependent on the ultrafilter or the filter device, it is desirably about 0.1-10.0 kg/cm². If necessary, the upper residue liquid treated may be diluted with suitable solvent such as water to be treated again, and the purity is increased.

The obtained solvent of the upper residue liquid may be removed by a common method such as concentration, freeze-drying and spray-drying to obtain powder of the neutralizing agent for vacuolating toxin. The solvent such as alcohol, acetone and acetonitrile may be concentrated under reduced pressure to be recovered and reused.

Such obtained neutralizing agent for vacuolating toxin is odorless powder of skin color, brown or pale yellow tasting little bitter. The agent adsorbs on synthetic resin having affinity for polyphenol, and it is proanthocyanidine which is not able to penetrate ultrafilter having a fraction molecular weight of 1,000 or more by treatment with the ultrafilter.

The yield is 0.5-20.0 w/w % converted to hop bract amount and 0.5-15.0 w/w % converted to hop cone.

As a method for obtaining the neutralizing agent for vacuolating toxin from apple, apple fruits, preferably unripe apple fruits is pressed the juice out, a solution of the neutralizing agent for vacuolating toxin is obtained, and the solution is concentrated, powder is obtained by a usual method such as freeze-drying or splay-drying. If necessary, the neutralizing agent for vacuolating toxin is purified with a column charging granular resin which has affinity for polyphenol to increase the purification. The process is operated by the same method as the process for concentrating and purifying the neutralizing agent for vacuolating toxin obtained from hop.

Thus obtained neutralizing agent for vacuolating toxin is able to formulate with a carrier, an auxiliary agent and an additive which are commonly used. The neutralizing agent may be used as an oral or parenteral pharmaceutical by a common method. It may be further used as food and drink, which are mixed with food materials.

As an oral pharmaceutical, there are tablets, capsules, granules, syrup and the like. As a parenteral pharmaceutical, there are external medicines such as ointments, creams and solutions, and injection such as sterilized liquid agents and suspending agents. When these products are given as a medicine to a human body, the amount of 2 mg-500 mg per day per one or several times, namely 2-1000 mg per all day is administered to obtain sufficient effects.

The medicines containing the neutralizing agent for vacuolating toxin may be a unit volume form that is desired along with physiologically recognizable vehicles, carriers, fillers, integrate agents, stabilizers, flavours and the like. Adjuvants mixed with tablets or capsules are as follows: a connective agent such as tragacanth, arabic gam, corn starch and gelatin, vehicle fillers such as microcrystal cellulose, explosive agents such as corn starch, all gelatinized starch, alginic acid, lubricants such as magnesium stearate, sweeteners such as sucrose, lactose and saccharin, flavors such as peppermint, acamono oil and cherry. The capsules may contain liquid carriers such as oils and fats in addition to the above-mentioned materials. The other materials may be used as covering agents for changing the physical forms by another method. For example, tablets may be covered with shellac or sucrose. Syrup or elixir may contain sucrose as a sweetener, methyl or propyl paraben as an antiseptic agent, a dye and a flavor such as a cherry or orange flavor.

A sterile composition for injection may be formulated by a common method that an active material in vehicle such as injection solvent, naturally produced vegetable oil such as sesame oil, coconut oil, peanut oil and cotton seed oil, and synthetic fat vehicle such as ethyloleate are dissolved or suspend. Further, if necessary, a buffer agent, an antiseptic agent, an antioxidant and the like may be compounded. A base such as Vaseline, paraffin, fats and fatty oils, lanolin and macrogol may be used for external medicines to obtain an ointment, a cream agent and the like by a common method.

Food and drink containing the neutralizing agent for vacuolating toxin of the present invention may be formed as shown in the above, or may be processed and prepared by a common method by addition of the neutralizing agent to each food materials in the form of wheat gluten, Japanese cracker, cookies, drink and the like. The food and drink can be ingested as health food or functional food for ill prevention and health maintenance several times and 5 mg-500 mg a day as processing products. When the neutralizing agent for vacuolating toxin is added to the food and drink, it may be added as powder, preferably as an aqueous solution, aqueous alcohol solution or alcohol solution containing 1-2% of the neutralizing agent. The addition amount is 1-10,000 ppm, preferably 100-5000 ppm of the final concentration.

The neutralizing agent for vacuolating toxin of the present invention may be used as a preventive agent for preventing digestive diseases, as a relapse preventive agent for preventing relapse of the digestive diseases, and as an agent for removing bacteria to treat the digestive diseases by removing pylori bacteria. When the digestive diseases is prevented, prevented relapse or treated, the neutralizing agent for vacuolating toxin may be used alone or combined with a proton pump inhibitor and/or an antibiotic.

The administration amount of the neutralizing agent for vacuolating toxin of the present invention may be suitably selected by usage, patient's age, gender and the other condition, and seriousness of the disease. Usually, the neutralizing agent for vacuolating toxin used as an effective ingredient may be administered about 0.1-2000 mg, preferably 0.5-1800 mg, most preferably 1.0-1500 mg a day by separating the amount 1-4 times a day at hungry time.

Preferred embodiments of this invention are disclosed in the following examples, but it is not intended to limit to these examples.

### EXAMPLE 1

### Preparation of the Neutralizing Agent for Vacuolating Toxin from Hop Cone with a Synthetic Adsorption Agent of a Gel Form

Hop cone 20 g was ground in a mortar, and extracted with water 2 L by stirring at a temperature of 95° C. for 40 minutes. After filtration, the extracted liquid was cooled, passed through a column packed hydrophilic vinyl polymer 80 ml, and the column was washed with a 5% ethanol aqueous solution 400 ml. An 80% ethanol aqueous solution 400 ml was passed through the column, the extracted solution was recovered, and freeze-dried to obtain the neutralizing agent for vacuolating toxin 800 mg as odorless powder of pale yellow tasting little bitter. The yield from the hop was 4%.

### EXAMPLE 2

### Preparation of the Neutralizing Agent for Vacuolating Toxin from Hop Bracts with a Synthetic Adsorption Agent of a Gel Form

Hop bracts 20 g was extracted with a 50% ethanol aqueous solution 600 ml by stirring at a temperature of 30° C. for 20 minutes. After filtration, the extracted liquid was concentrated under reduced pressure. The concentrated liquid was passed through a column packed styrene-divinyl benzene resin 80 ml, and the column was washed with water 400 ml. An 80% ethanol aqueous solution 400 ml was passed through the column, the solution was recovered, and freeze-dried to obtain the neutralizing agent for vacuolating toxin 1.6 g as odorless powder of pale yellow tasting little bitter. The yield from the hop bracts was 8%.

### EXAMPLE 3

### Preparation of the Neutralizing Agent for Vacuolating Toxin from Hop Cone with Ultra Filter

Hop cone 20 g was ground in a mortar, and extracted with water 2 L by stirring at a temperature of 95° C. for 40 minutes. After filtration, the extracted liquid was cooled, and treated with ultra filter of fraction molecular weight 50,000 at 1.0 kg/cm² at room temperature to obtain 20 ml. The obtained upper residue liquid was dried under reduced pressure to obtain the neutralizing agent for vacuolating toxin 200 mg as odorless powder of pale yellow tasting little bitter. The yield from the hop was 1%.

### EXAMPLE 4

### Preparation of the Neutralizing Agent for Vacuolating Toxin from Hop Bracts with Ultra Filter

Hop bracts 20 g was extracted with a 50% ethanol aqueous solution 600 ml by stirring at a temperature of 80° C. for 40 minutes. After filtration, the extracted liquid was treated with ultra filter of fraction molecular weight 1,000 at 3.0 kg/cm² at room temperature to obtain 60 ml. The obtained upper residue liquid was freeze-dried to obtain the neutralizing agent for vacuolating toxin 0.8 g as odorless powder of pale yellow tasting little bitter. The yield from the hop bracts was 4%.

### EXAMPLE 5

### Further Purification of the Neutralizing Agent for Vacuolating Toxin and the Qualitative Analysis

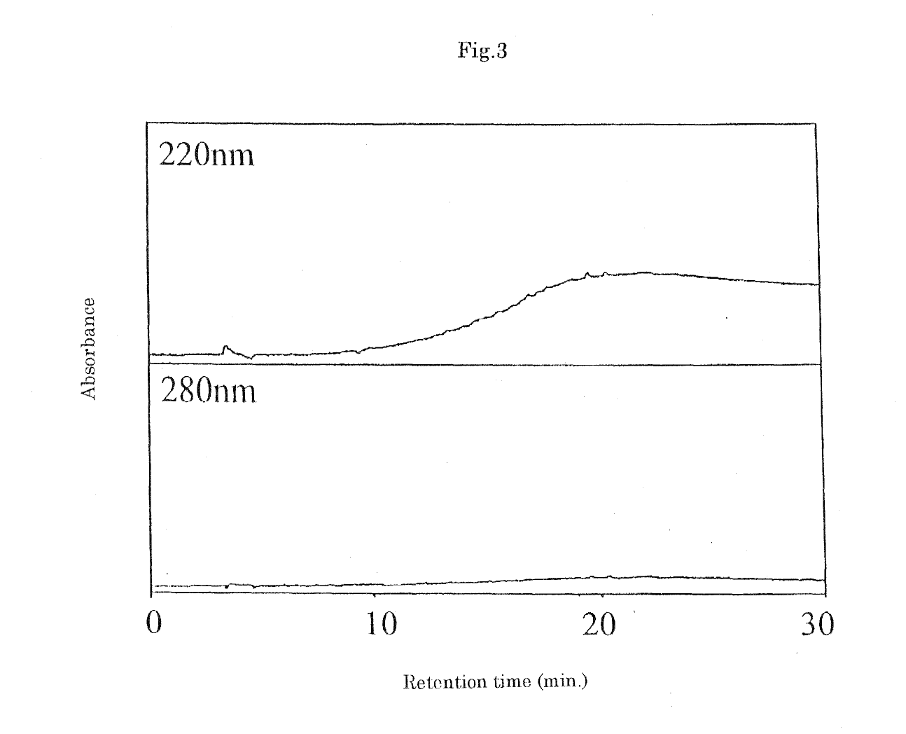

The neutralizing agent for vacuolating toxin 0.8 g obtained by Example 2 was dissolved in a 10% ethanol aqueous solution 500 ml, and treated with ultra filter of fraction molecular weight 5,000 at 1.0 kg/cm² at room temperature to obtain 20 ml. The obtained upper residue liquid was freeze-dried to obtain the neutralizing agent for vacuolating toxin 0.4 g as odorless powder of skin color tasting little bitter. The power was analyzed by HPLC. The result was shown by a characteristic chromatogram of FIG. 3. By catechin quantitative determination that is a common quantitative method of polyphenols, it was 40.6% reduced to catechin.

(HPLC conditions) Device: Shimazu LC-10A system, column: ODS-80™ (Toso, 4.6 mmI.D.×25 cm), movement phase: straight gradient for 30 minutes of (liquid A, liquid B)=from (100.0) to (50:50), liquid A: 5% acetonitrile (containing 0.1% HCl), liquid B: acetonitrile, sample injection amount: 20 μg, detection: multiwavelength at 200-300 nm.

### EXAMPLE 6

### Preparation of the Neutralizing Agent for Vacuolating Toxin from Immature Apple Fruits

Immature apple fruits (average weight 5.03 g) 400 g was homogenized with 1% hydrochloric acidic methanol, and extracted under heat refluxing (three times). The extracted solution was concentrated under reduced pressure to remove methanol, and chloroform was added to distribute the solution (two times). Water phase was recovered and filtered, and distilled water was added to obtain 200 ml. The solution was purified by an extracting method of solid phase with Sep-pak C18, and freeze-dried to obtain the neutralizing agent for vacuolating toxin.

### EXAMPLE 7

(Tablets and Capsules)

| | | |
|---|---|---|
| Material obtained by Example 5 | 10.0 | g |
| Lactose | 75.0 | g |
| Magnesium stearate | 15.0 | g |
| Total | 100.0 | g |

These were homogeneously mixed to obtain tablets and capsules by a common method. The same method was conducted except that materials obtained by Examples 1, 2, 3, 4 and 6 were used instead of using the materials obtained by Example 5.

### EXAMPLE 8

(Powder and granule)

| | | |
|---|---|---|
| Material obtained by Example 5 | 20.0 | g |
| Starch | 30.0 | g |
| Lactose | 50.0 | g |
| Total | 100.0 | g |

These were homogeneously mixed to obtain powder and granule by a common method. The same method was conducted except that materials obtained by Examples 1, 2, 3, 4 and 6 were used instead of using the materials obtained by Example 5.

### EXAMPLE 9

(Injection)

| | | |
|---|---|---|
| Material obtained by Example 5 | 1.0 | g |
| Detergent | 9.0 | g |
| Physiological saline | 90.0 | g |
| Total | 100.0 | g |

These were heat-mixed and sterilized to obtain injection. The same method was conducted except that materials obtained by Examples 1, 2, 3, 4 and 6 were used instead of using the materials obtained by Example 5.

### EXAMPLE 10

(Wheat gluten)

| | | |
|---|---|---|
| Sucrose | 20.0 | g |
| Thick malt syrup (75% solid) | 70.0 | g |
| Water | 9.5 | g |
| Coloring matter | 0.45 | g |
| Flavor | 0.045 | g |
| Material obtained by Example 5 | 0.005 | g |
| Total | 100.0 | g |

These were heat-mixed and sterilized to obtain wheat gluten. The same method was conducted except that materials obtained by Examples 1, 2, 3, 4 and 6 were used instead of using the materials obtained by Example 5.

### EXAMPLE 11

(Juice)

| | | |
|---|---|---|
| Concentrated orange juice | 15.0 | g |
| Fructose | 5.0 | g |
| Citric acid | 0.2 | g |
| Flavor | 0.1 | g |
| Coloring matter | 0.15 | g |
| Sodium ascorbate | 0.048 | g |
| Material obtained by Example 5 | 0.002 | g |
| Water | 79.5 | g |
| Total | 100.0 | g |

These were heat-mixed and sterilized to obtain juice. The same method was conducted except that materials obtained by Examples 1, 2, 3, 4 and 6 were used instead of using the materials obtained by Example 5.

### EXAMPLE 12

(Cookies)

| | | |
|---|---|---|
| Soft flour | 32.0 | g |
| Whole egg | 16.0 | g |
| Butter | 16.0 | g |
| Sucrose | 25.0 | g |
| Water | 10.8 | g |
| Baking powder | 0.198 | g |
| Material obtained by Example 5 | 0.002 | g |
| Total | 100.0 | g |

These were mixed to obtain cookies by a common method. The same method was conducted except that materials obtained by Examples 1, 2, 3, 4 and 6 were used instead of using the materials obtained by Example 5.

### EXAMPLE 13

### Cell Toxicity Test of Culture Cells for the Vacuolating Toxin

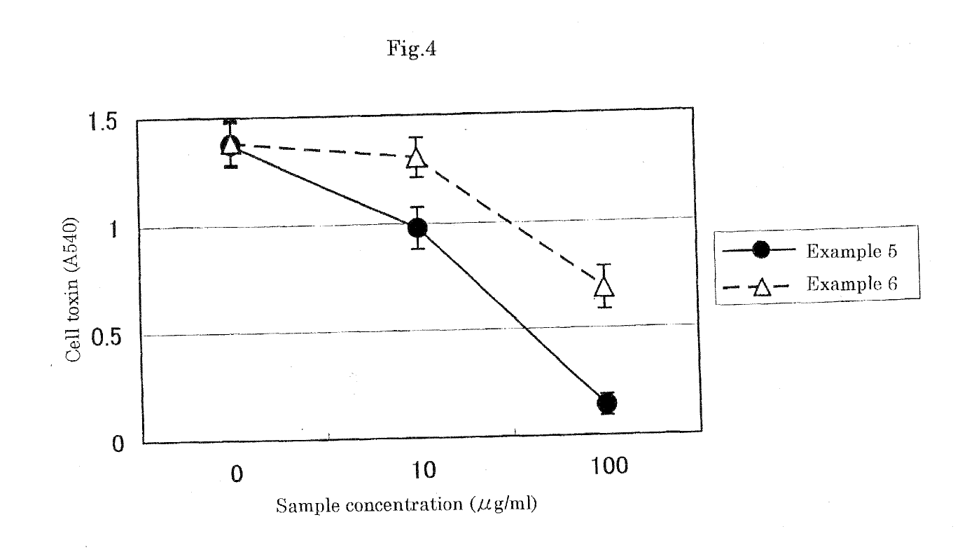

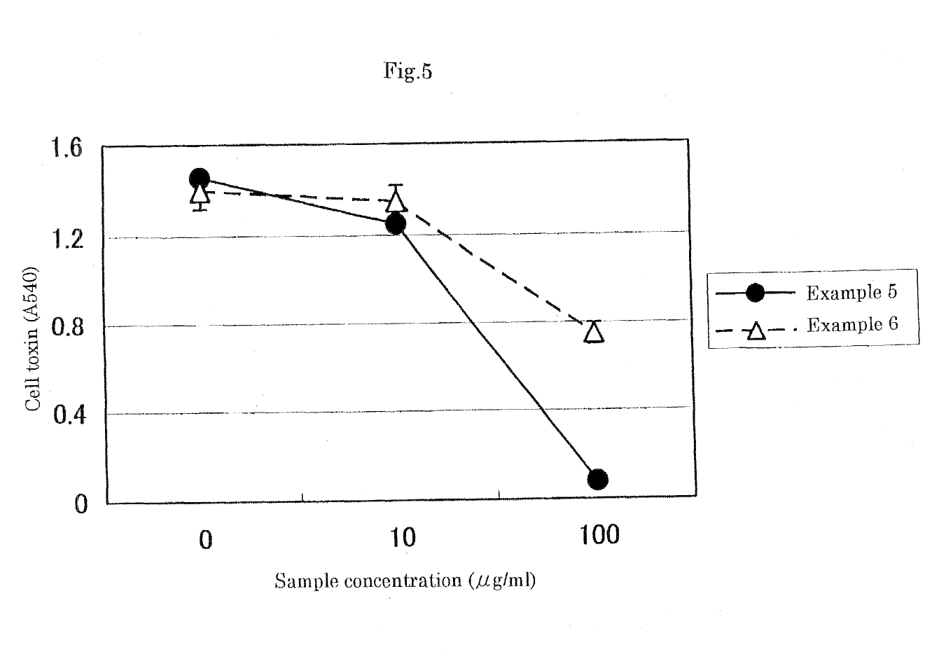

AZ-521 cells derived from human stomach cancer or G401 cells derived from human kidney cancer were prepared to obtain 2.0×10⁵ cells/ml of suspension. The suspension 100 μl was injected into each hole of a 96 hole plate, and left overnight to obtain monolayer membrane of each cell. A certain concentration of the vacuolating toxin and multiple concentration of the neutralizing agent for vacuolating toxin obtained by Example 5 or 6 were mixed. The mixture was incubated at a temperature of 37° C. for 30 minutes, and added to the holes. The final concentration of the vacuolating toxin was 120 nM, and the final concentration of materials of Example 5 or 6 was 0-100 μg/ml. The cells in the plate were cultured at a temperature of 37° C. for 8 hours under a 5% CO₂ atmosphere. The toxicity of the vacuolating toxin for the cells was evaluated by uptake degree (Ab540) to the vacuole of neutral red (a 0.05% PBS solution). The results are shown in FIG. 4 and FIG. 5. It shows that, depending on the concentration of the neutralizing agents for vacuolating toxin obtained by Examples 5 and 6, the cell toxicity of the vacuolating toxin was nontoxic to the both of AZ-521 cell and G401 cell.

### EXAMPLE 14

### Binding to Culture Cells

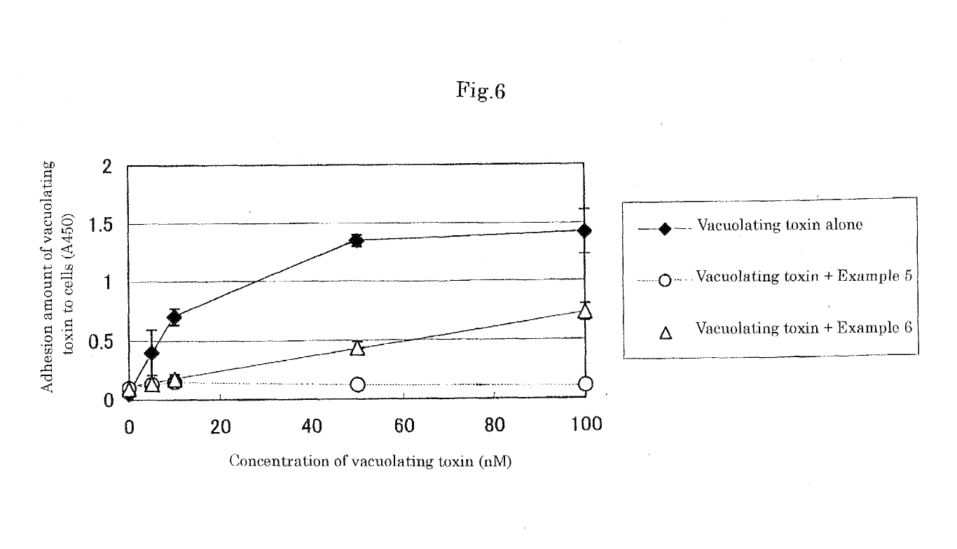

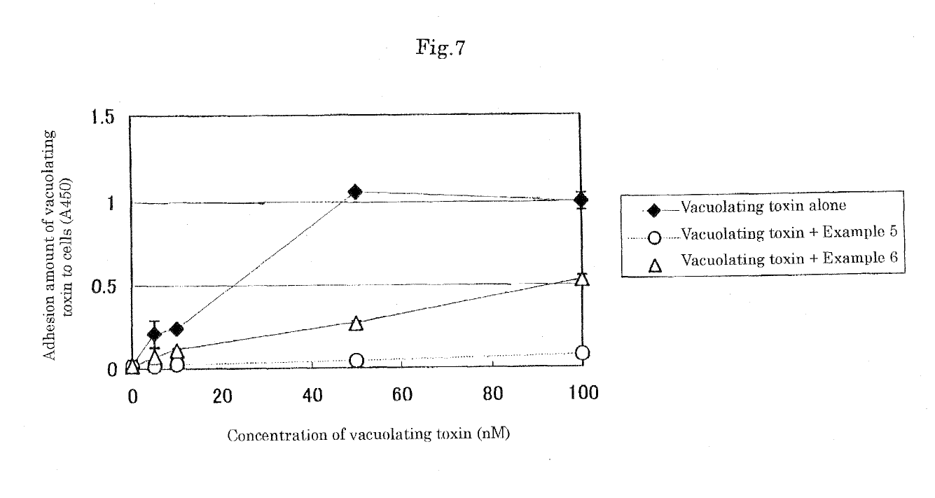

AZ-521 cells derived from human stomach cancer or G401 cells derived from human kidney cancer were prepared to obtain 2.0×10⁵ cells/ml of suspension. The suspension 100 μl was injected into each hole of a 96 hole plate, and left overnight to obtain monolayer membrane of each cell. Multiple concentrations of biotin labeling vacuolating toxin and a certain concentration of neutralizing agent for vacuolating toxin obtained by Example 5 or 6 were incubated at a temperature of 37° C. for 30 minutes, and added to the monolayer membrane of each cell. The final concentration of the vacuolating toxin was 0-100 nM, and the final concentration of materials of Example 5 or 6 was 10 μg/ml. The monolayer membranes of the cells were cultured in an incubator at a temperature of 37° C. for 4 hours under a 5% CO₂ atmosphere and then the cells were fixed with 0.25% glutaraldehyde. The amount of the biotin-labeled vacuolating toxin adhered to cell surfaces was evaluated with avidin-labeled horseradish peroxidase (Pharmacia) and coloring (Ab450 nm) of a TMBZ dye. The results are shown in FIG. 6 and FIG. 7. It shows that, depending on the concentration of the neutralizing agents for vacuolating toxin obtained by Examples 5 and 6, the binding to the cells of vacuolating toxin was inhibited.

### EXAMPLE 15

### Mouse Stomach Damage Test

The vacuolating toxin 5 μg per 10 g body weight and the materials of Example 5 50-250 μg were administered with an ingestion probe to C57BL/6J mice of 4 week old that was fasted for 24 hours (only water was drunk freely). The mice were bred in each cage. After 48 hours, the stomachs were extirpated. The extirpated sample was fixed with 10% formalin, and observed with a stereoscopic microscope before and after the fixation. The fixed sample was stained with hematoxilin eosin, according as a method of Ghiara (Ghiara, P., et al., Infect. Immun., 63, 4154-4160 (1995)), the degree of stomach damage was evaluated and counted. The results are shown in Table 1. The materials of Example 5 inhibited the stomach damage significantly.

TABLE 1

| | | Score of |
|---|---|---|
| Number | Sample | stomach damage |
| 1 | phosphate buffer | 1.6 ± 0.8 |
| 2 | Example 5 (250 μg) | 1.8 ± 0.8 |
| 3 | vacuolating toxin (5 μg) | 3.0 ± 0.8 |
| 4 | vacuolating toxin (5 μg) + | 2.4 ± 1.0 |
| | Example 5 (50 μg) | |
| 5 | vacuolating toxin (5 μg) + | 2.2 ± 0.8* |
| | Example 5 (100 μg) | |
| 6 | vacuolating toxin (5 μg) + | 2.2 ± 0.8* |
| | Example 5 (250 μg) | |

*significantly difference comparing with 3 is a critical value 5% or less.

### INDUSTRIAL APPLICABILITY

The neutralizing agent for vacuolating toxin of the present invention has an effect of attenuating the vacuolating toxin, so that the agent has an effect of preventing and treating the infection diseases caused by the vacuolating toxin. The product of the present invention can be prepared as preventive/therapeutic agents for the infective diseases caused by the vacuolating toxin.

The digestive diseases participated by pylori are exemplified by gastric ulcer, duodenal ulcer, gastritis, gastric cancer and MALT lymphocytoma.

1. A proanthocyanidine having an effect of attenuating a vacuolating toxin produced by Helicobacter pylori, wherein the proanthocyanidine originates in hop or hop bract.

2. A treating agent composition for gastritis, gastric ulcer or gastric cancer in which Helicobacter pylori participates, comprising an effective amount of the proanthocyanidine as claimed in claim 1, and a pharmaceutically acceptable carrier or excipient.

3. A neutralizing agent for vacuolating toxin that is produced by Helicobacter pylori, comprising an effective amount of the proanthocyanidine as claimed in claim 1, and a pharmaceutically acceptable carrier or excipient.

4. A food and drink product containing as an effective ingredient the proanthocyanidine as claimed in claim 1.

5. A pharmaceutical composition for attenuating a vacuolating toxin produced by Helicobacter pylori, comprising an effective amount of 2-1000 mg of a proanthocyanidine originating in hop or hop bract, and a pharmaceutically acceptable carrier or excipient.

6. A proanthocyanidine derived from hop bracts and having a property of being capable of attenuating a vacuolating toxin produced by Helicobacter pylori.

7. The proanthocyanidine according to claim 1 which is prepared by:
a. extracting hop bracts with an aqueous solution of ethanol;
b. passing the extracted liquid through a column packed with styrene divinyl benzene resin;
c. passing an aqueous ethanol solution having a higher concentration of ethanol than the aqueous solution of ethanol in step a; and
d. freeze drying the resulting solution.
